(19) United States
(12) Patent Application Publication
(10) Pub. No.: US 2024/0393311 A1
(43) Pub. Date: Nov. 28, 2024

(54) METHOD FOR DETERMINING A QUALITY OF A MOLASSES USED IN YEAST PRODUCTION

(71) Applicant: LESAFFRE ET COMPAGNIE, PARIS (FR)

(72) Inventors: Melvyn ALLAIRE, MONTEVRAIN (FR); Mathieu CLEMENT-ZIZA, LAMBERSART (FR); Ludovic DELINE, WASQUEHAL (FR)

(73) Assignee: LESAFFRE ET COMPAGNIE, PARIS (FR)

(21) Appl. No.: 18/694,609

(22) PCT Filed: Sep. 23, 2022
(86) PCT No.: PCT/FR2022/051797

§ 371 Date: Mar. 22, 2024

(30) Foreign Application Priority Data
Sep. 24, 2021 (FR) 2110103

(51) Int. Cl. G01N 33/02; C12N 1/16; G01N 21/31; G01N 22/00

(52) U.S. Cl. CPC G01N 33/02; C12N 1/16; G01N 21/31; G01N 22/00

(57) ABSTRACT

A method for qualifying molasses based on an optical measurement, the method may include: based on reference optical spectra of samples of distinct molasses, associated with respective known molasses qualities, building by machine learning a statistical model of molasses qualities as a function of at least one spectral characteristic of the reference spectra; and for a current optical spectrum of a sample of a current molasses, based on the statistical model, identifying the at least one spectral characteristic of the current optical spectrum and determining a quality of the current molasses, the current molasses quality relating to at least one yeast performance obtained when the yeast is fed with the current molasses.

### FIELD

The present disclosure relates to the field of yeast production, and more particularly, it relates to a method for determining a quality of a molasses used as a source of nutrients in the production of a yeast, this molasses quality relating to one or more performance(s) of the produced yeast.

### BACKGROUND

The term “yeast”, in the singular or in the plural, is a generic term referring to eukaryotic microorganisms capable of causing a fermentation of organic materials. Yeasts are commonly used, whether by individuals or by the agri-food or pharmaceutical industry. For example, they may be used in the manufacture of wine, beer, or leavened doughs (for example breads). Among yeasts, mention will be made, in a non-exhaustive manner, of the genera Saccharomyces, Candida, Pichia, Kluyveromyces. The expression “yeast strain” refers to a relatively homogeneous population of yeast cells. A yeast strain is obtained from the isolation of a clone. A clone gives birth to a population of cells obtained from one single yeast cell.

A yeast is generally produced based on the multiplication of a strain cultured in a medium comprising a set of raw materials. In an industrial environment, based on a given starting amount of yeast, it is possible to produce several tons at the output. The raw materials generally used to feed yeast production consist of a set of nutrients and a source of sugar(s), which may consist of a sugar syrup, molasses, a mixture of sugar syrups and/or molasses.

The molasses is a raw material derived from the sugar industries and it may be distinguished according to two types of molasses, beet molasses or cane molasses. The nutrients may be sources of nitrogen, phosphorus, minerals, trace elements (for example Fe, Zn, etc.), vitamins (for example B, C, etc.), water, or air.

During the production of yeast, several factors could affect the yield or the quality of the produced yeast. These factors may consist of the quality of the used molasses as well as the different proportions of nutrients added to the yeast. Depending on a given molasses quality or other factors or a set of factors including the quality of the molasses, it is possible to adjust the production schemes like, for example, the respective amounts of each nutrient so as to optimise the production yield and/or the quality of the yeast at the output.

Currently, the quality of a molasses that has not yet been used in production as well as the quality of the yeast produced based on this molasses can be determined only after either a first yeast production followed by qualification protocols of the produced yeast, or a series of miniaturised tests in the laboratory allowing estimating the quality roughly and separating the atypical molasses qualities. Indeed, the quality of an unknown molasses or the compensation for a known, yet poor-quality, molasses by adjusting the production schemes can be done only after a qualification of the yeast obtained based on this molasses quality.

Knowing that a first production might correspond to several hundred tons of yeast produced and might have required the operation of several production sites, obtaining a poor production yield or a poor-quality yeast could represent a significant loss for an industrial.

In parallel, the organisation of the production of the molasses in “sugar campaigns” leads to the fact that the quality of the molasses received over time might feature sensitive variations, and that it is not possible to anticipate the impact of these variations.

These variations in quality and their impact on the quality of the yeast are even less easy to anticipate as the molasses are stored in tanks that could contain quantities that can be used for several weeks of production, which makes it even more difficult to detect a batch of molasses with the atypical quality that would have been transferred into the storage tank.

Hence, there is a need to determine the quality of a molasses before use thereof in yeast production so as to avoid the production of a yeast having a non-compliant (for example with a specification set) quality and/or performances.

### SUMMARY

The present disclosure improves the situation.

Thus, a method for qualifying molasses based on an optical measurement is proposed, the method may comprise:
based on reference optical spectra of samples of distinct molasses, associated with respective known molasses qualities, building by machine learning a statistical model of molasses qualities as a function of at least one spectral characteristic of the reference spectra, and
for a current optical spectrum of a sample of a current molasses, based on the statistical model, identifying said at least one spectral characteristic of the current optical spectrum and determining a quality of the current molasses, said current molasses quality relating to at least one yeast performance obtained when said yeast is fed with said current molasses.

Advantageously, it is thus possible to determine the quality of an unknown molasses based on its optical spectrum, and to deduce therefrom at least one yeast performance of the yeast produced based on this unknown molasses for production conditions (for example temperature and pressure in the fermenter) and a determined yeast strain.

Thus, advantageously, a molasses that does not have the quality required to allow obtaining a yeast having one or more target performance(s) could be discarded before any use in production. According to another advantage, based on the determined molasses quality, it is possible to adapt the production schemes like, for example, by adjusting the proportions of nutrients necessary in order to correct a poor-quality molasses so as to enable the target performance(s). Furthermore, depending on the degree of poor quality of a molasses, it may be possible to correct this quality by mixing the molasses with molasses of different qualities.

In one or more embodiment(s), each molasses quality associated respectively with a distinct molasses sample relates to at least one yeast performance obtained when said yeast is fed with said distinct molasses sample.

In one or more embodiment(s), said at least one performance relating to a molasses quality associated respectively with a distinct molasses sample relates to a reference yeast obtained when said reference yeast is fed with said distinct molasses sample. In one or more embodiment(s), the molasses quality of a distinct molasses sample is defined by at least one molasses quality score, said at least one molasses quality score relating to said at least one yeast performance.

By reference yeast, it may be understood a reference yeast used in a qualification protocol having determined at least one performance of said reference yeast, said reference yeast having been fed with said distinct molasses sample.

Optionally, the features disclosed in the next paragraphs may be implemented. They may be implemented independently of one another or in combination with one another.

According to one or more embodiment(s), said at least one performance is based on at least one criterion selected from among:
a multiplication capacity of the yeast fed with said current molasses,
a bread leavening capacity,
a capacity to be preserved,
a capacity to be dried,
a capacity to fix structural elements,
a capacity to assimilate nutrients,
a capacity to adapt to stresses.

By multiplication capacity of the yeast fed with said current molasses, it may be understood a production yield per unit of current molasses mass implemented or a ratio of the amount of yeast produced to the amount of sugar used in the production process.

By structural elements, it may be understood elements such as nitrogen, phosphorus, carbon, hydrogen, oxygen or sulphur.

In one or more embodiment(s), the quality of the current molasses determined by said statistical model is defined by at least one molasses quality score, said at least one molasses quality score relating to said at least one performance.

In one or more embodiment(s), rather than using a score or in combination with a score, the quality of the current molasses determined by said statistical model may be defined by a class such as good, bad, medium, very good, green light to use this current molasses, red light to discard, or modify this current molasses, etc., the class relating to at least one performance of the yeast obtained when it is fed with this current molasses.

In one or more embodiment(s), the method may further comprise:
illuminating a molasses sample with a first emitted light signal interacting with the sample, and collecting a second light signal, resulting from the interaction between the first light signal and the current sample; and
measuring the second light signal, in selected frequency bands, to establish an optical spectrum representative of a chemical signature of the current molasses sample.

In one or more embodiment(s), the established optical spectrum and the reference optical spectra may be measured under similar conditions of illumination and measurement of light signals.

Thus, for example, in one embodiment, the statistical model may be determined by implementing a machine learning, for example based on a random forest to end with a set of decision trees defining the statistical model.

In one or more embodiment(s), the optical spectrum may comprise a set of wavenumbers, one or more wavenumber(s) of the set of wavenumbers being representative of a chemical compound of the molasses.

According to one or more embodiment(s), the molasses may include compounds at least one from among a sugar, a mineral, a vitamin, and the optical spectrum includes a plurality of wavenumbers, and wherein at least one wavenumber characterises the presence of one of said compounds.

According to one or more embodiment(s), said at least one spectral characteristic may be determined according to at least one criterion selected from among a presence or an absence of a predefined wavenumber, a position of a predefined number, an intensity value of a predefined wavenumber, an overpassing of threshold of an amplitude of a predefined wavenumber, a maximum or minimum intensity value of a predefined wavenumber, an intensity deviation between two predefined wavenumbers, an intensity of a predefined wavenumber comprised between two predetermined intensity values, or simple or complex combinations of the aforementioned spectral characteristics.

In one or more embodiment(s), said at least one spectral characteristic may be determined according to at least one criterion selected from among a presence or an absence of a predefined line, a position of a predefined line, an intensity value of a predefined line, overpassing of a threshold of an amplitude of a predefined line, a maximum or minimum intensity value of a predefined line, an amplitude deviation between two predefined lines, an amplitude of a predefined line comprised between two predetermined amplitude values, or simple or complex combinations of the aforementioned spectral characteristics.

According to one or more embodiment(s), the optical measurement may be carried out by at least one technique from among a RAMAN spectroscopy, an IR spectroscopy, or a THZ spectroscopy.

According to one or more embodiment(s), the range of values of the reference optical spectra may be comprised between 1 cm⁻¹ and 2,500 cm⁻¹.

According to one or more embodiment(s), the range of values of the reference optical spectra may be comprised between 436 cm⁻¹ and 1,700 cm⁻¹.

According to one or more embodiment(s), the molasses is a cane molasses or a beet molasses or a mixture of both, or a mixture of sugar products such as a molasses and a sugar syrup, or a mixture of products one of the components of which is the molasses.

The present disclosure also relates to a computer program comprising program code instructions for executing the method of the present disclosure when the program could be executed on a computer.

The present disclosure also relates to a non-transitory computer-readable recording medium on which a program is recorded for implementing the method of the present disclosure when this program is executed by a processor.

The present disclosure also relates to a method for producing yeasts by fermentation which may comprise:
determining a respective molasses quality for each molasses of a plurality of molasses supplied at the input of the fermentation reaction according to the molasses qualification method described in the present disclosure, and
feeding the fermentation reaction with each molasse of the plurality of molasses whose respective molasses quality is higher than a predetermined quality, and for all or part of the molasses having a molasses quality lower than the predetermined quality, feeding the fermentation reaction after modification of the molasses, or/and discarding the molasses.

By modifying the molasses, in one or more embodiment(s), it is possible to consider at least one modification selected from among:
mixing the molasses having a molasses quality lower than the predetermined quality, with another molasses having a distinct molasses quality, and so that the molasses mixture has a molasses quality higher than the predetermined quality,
adding one or more additive(s) so that the molasses improved by the additives has a molasses quality higher than the predetermined quality.

The respective molasses quality of each molasses of the plurality of molasses and which relates to at least one performance of the obtained yeast can be determined for identical fermentations conditions (for example temperature and pressure of the fermenter, fermenter type, etc.) and for the same yeast strain. Brief description of the drawings.

### BRIEF DESCRIPTION OF THE DRAWINGS

Other features, details and advantages will become apparent upon reading the detailed description hereinafter, and upon analysis of the appended drawings, wherein:

FIG. 1 schematically describes, In one or more embodiment(s), an example of an optical spectrum of a molasses.

FIG. 2 illustrates a method for determining a quality of a molasses based on an optical measurement in one or more embodiment(s).

FIG. 3 illustrates an example of application of the statistical model obtained according to the method of the present disclosure on spectra of unknown molasses.

FIG. 4a illustrates the principle of random forest for a statistical model in one or more embodiment(s).

FIG. 4b illustrates, the principle of random forest for a statistical model in one or more embodiment(s).

FIG. 5 shows different spectra of cane molasses of known quality obtained by RAMAN spectroscopy.

FIG. 6 shows a device for implementing the method of the present disclosure according to one or more embodiment(s).

### DETAILED DESCRIPTION

The terms “peak” and “line” may be interpreted in the same manner and are interchangeable in the present disclosure. Similarly, the terms “intensity” and “amplitude” may be interpreted in the same manner and are interchangeable in the present disclosure.

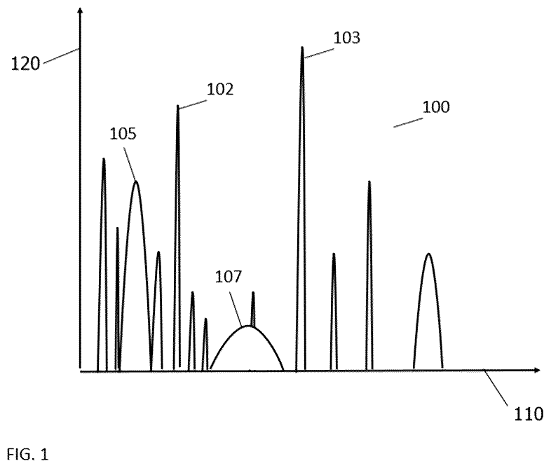

FIG. 1 schematically describes, in one or more embodiment(s), an example of an optical spectrum of a molasses.

There are many optical measurement techniques allowing obtaining an optical spectrum, like, for example, infrared, microwave, Terahertz spectroscopy, or RAMAN spectroscopy. All these techniques are based on the same principle consisting in sending an electromagnetic wave (i.e. photons) on the material to be sensed by varying the frequency of the wave. Afterwards, a wave resulting from the wave-material interaction may be collected, either in transmission or in reflection, for each frequency of the electromagnetic wave. Based on the resulting wave, it is then possible to determine an optical spectrum representative of the constituents of an organic material (for example a molasses), i.e. a chemical signature of the molasses.

An optical spectrum 100 may be represented as a set of peaks, where each peak may be associated with a position (for example abscissa) and an amplitude (for example ordinate) in the spectrum. The abscissa 110 of an optical spectrum may be expressed either in frequency (Hz), or in wavelength or in wavenumber (cm-1). The ordinate 120 may be expressed in an arbitrary unit which may depend on the signal processing applied on the measurement data. Indeed, in order to be able to be correctly exploitable, it is common that one or more data processing operation(s) is/are applied on the raw data (i.e. data derived from the optical measurements) in order to enable the comparison between the spectra for example.

For example, in the case of RAMAN spectroscopy, a baseline, a derivation of this baseline or a combination with the normalisation of the data may be carried out in order to eliminate some peaks having aberrations or the presence of which results from the Laser used for the spectroscopy,

A specific range of frequencies (or wavelengths or wavenumbers) of use in which the frequency of the incident wave varies may correspond to each optical measurement technique. For example, the range of wavelengths (expressed in microns) may be comprised between [1 μm-20 μm], [0.3 μm-0.7 μm], or else [1 cm⁻¹-2,500 cm⁻¹]. In one or more embodiment(s), this frequency range may be located between [436 cm⁻¹-1,700 cm⁻¹].

Depending on the optical measurement technique and the wavelength range used to obtain a spectrum of an organic material, the position of a peak may correspond, for example, either to an atomic or chemical bond, or to a functional group of an organic molecule, or the vibrations of a molecule. Hence, several peak positions may correspond to the presence of the same molecule (or constituent, for example, a vitamin) in an organic material by reflecting, for example, the chemical bonds or functional groups of this molecule.

The amplitude of a peak may have different meanings depending on the used optical measurement technique and the data processing applied on the raw optical data.

The optical measurement techniques allowing carrying out the spectroscopy of a medium are generally complementary, and the same medium may be measured by different optical measurement techniques (for example different types of spectrometers) in order to obtain a relatively complete mapping of this medium.

Depending on the used measurement techniques, the exploitation of spectra may be more or less complicated whether visually or according to conventional methods for exploiting spectral data. Indeed, an optical spectrum may comprise a multitude of peaks (or lines), some of which could be perfectly discretised 102; 103, whereas others may be so close so to form a set 105; 107 making exploitation thereof difficult. Furthermore, depending on the used resolution (for example the step of the optical measurement), a peak dependent on its half-height width could spread more or less over an optical spectrum, and thus could complicate a little more the interpretation of the data.

Furthermore, while a change in the used optical measurement technique might seem to be obvious when the obtained optical spectrum cannot be exploited (for example a line cluster 107), the use of another optical measurement technique could lead to obtaining an optical spectrum completely or partially different from the spectrum obtained initially. Indeed, it is possible that the desired compounds/molecules are not sensitive to the wavelengths used by an alternative optical technique.

Thus, it could therefore be crucial to be able to exploit an optical spectrum, independently of the configuration of the peaks of the spectrum, and more particularly when interpretations and comparisons between a large number of optical spectra obtained based on relatively close samples in their chemical/molecular footprint are desired.

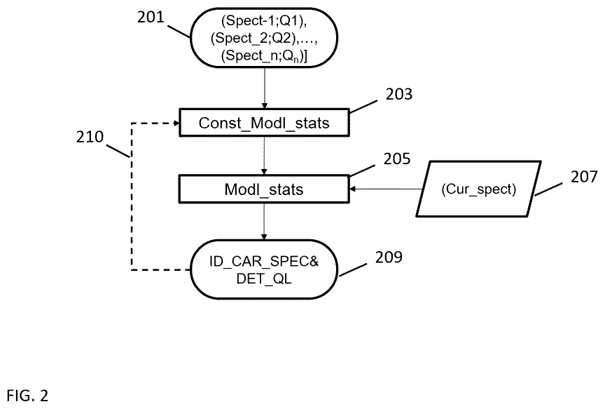

FIG. 2 illustrates a method for determining a quality of a molasses based on an optical measurement in one or more embodiment(s).

Based on reference optical spectra (Spect_ref1 . . . Spect_refn, n being a positive integer) 201 of samples of distinct molasses which are known and which are associated respectively with known molasses qualities (Q1, Q2, Qₙ), a statistical model 203 for qualifying molasses according to at least one spectral characteristic of the reference spectra may be built by machine learning.

The machine learning may consist in determining, in the reference spectra of molasses, spectral configurations leading to a certain molasses quality, in particular certain molasses qualities suited to the obtainment of one or more desired performance(s) for a yeast for example.

Based on the determined spectral configurations, the machine learning can then build one or more rule(s) which, together, form a statistical model correlating one or more spectral characteristic(s) of the spectrum to a molasses quality, this molasses quality relating to one or more performance(s) of a yeast produced using this molasses quality.

The reference spectra and the respective qualities of the molasses associated with the reference spectra may correspond to input data for feeding the machine learning, and each reference spectrum may be representative of a structure (for example chemical) of a molasses (for example cane or beet molasses). Furthermore, each molasses quality presented as input data and associated respectively with a reference spectrum could relate to at least one yeast performance (for example reference yeast) obtained when this yeast is fed with this molasses.

Furthermore, in one or more embodiment(s), the molasses quality may be defined by at least one respective score Scₙ and/or according to a molasses class (for example good, bad, average, red light, green light, etc.) and/or a correlation between its spectral profile and a statistical group of industrial performances.

In one or more embodiment(s), the molasses qualification score may relate to the impact of the quality of a molasses, when the latter is used as a source of nutrients in a yeast production, on the performance(s) of the obtained yeast.

For example, the score of each molasses associated with a reference spectrum may have a value comprised between 0 and 1 (for example, the value 0=bad and the value 1=good molasses) or 1 and 10. The value 1 may be representative of a poor-quality molasses and the value 10 representative of a very good-quality molasses. Thus, a score (or a score value) higher than 8 may correspond to a molasses allowing obtaining a yeast with very good performances, for example a very good bakery dough, such as bread, leavening capacity and/or a very good multiplication capacity (i.e. a good yeast production yield).

In one or more embodiment(s), a molasses quality may be defined according to a range of values. For example, a score comprised between 0 and 3 may correspond to a poor-quality molasses, a score comprised between 3 and 5 may correspond to a medium-quality molasses, a score comprised between 5 and 8 may correspond to a good-quality molasses, and a score higher than 8 on a scale of 10 may correspond to a very good-quality molasses.

According to another example, in one or more embodiment(s), the molasses quality may be defined by a plurality of scores, each score relating to a respective performance. For example, a reference optical spectrum may be associated with a first score relating to a bread leavening capacity, and to a second score relating to a multiplication capacity (i.e. yeast production yield).

The scores may be combined with one another in order to derive information that could be exploited by the personnel responsible for the production of the yeast within an industry or a test laboratory.

The score of each molasses associated with a reference spectrum may have been determined beforehand based on one or more qualification protocol(s).

For example, a protocol for qualifying a molasses may consist in the implementation of this molasses with a reference yeast in order to determine the performances of the reference yeast, in terms of cell multiplication and enzymatic activity.

For example, the performance based on (or relating to) the bread leavening capacity may be assessed by the capacity of the prepared yeast (for example the prepared reference yeast) to produce carbon dioxide gas, and/or may be assessed by the fermentative activity of the yeast.

The fermentative activity is determined by measuring the carbon dioxide gas release (expressed in an absolute manner as a volume or in a relative manner as a percentage) of a given dough using for example a Burrows and Harrison fermentometer described in the “Journal of Institute of Brewing”, vol. LXV, No. 1, January-February 1959 or a Risograph according to the protocol described by Rattin et al. 2009 (Cereal Foods World, 54 (6): 261-265).

In one or more embodiment(s), a molasses quality score Scn higher than 5 (i.e. corresponding to a good-quality molasses for example), may relate to a bread leavening capacity (i.e. yeast performance) in comparison with that generally observable on the yeasts produced in a reference method. Similarly, a molasses quality score Scn lower than 3 (i.e. corresponding to a poor-quality molasses), may relate to a bread leavening capacity allowing reaching specific volume values lower than the values generally observable on the yeasts produced in a reference method.

The multiplication capacity of the yeast fed with a current molasses, also called production yield, may be expressed as an amount of yeast produced for an amount of sugar used, or as the amount of sugar used per amount of yeast produced.

In one or more embodiment(s), a molasses quality score Scn higher than 5 (i.e. corresponding to a good-quality molasses for example), may relate to a yeast multiplication capacity (i.e. a yeast performance) that could generally be observed on the yeasts produced in a reference method. Similarly, a molasses quality score Scn lower than 3 (i.e. corresponding to a poor-quality molasses), may relate to a multiplication capacity of the yeast allowing reaching values lower than the values generally observable on the yeasts produced in a reference method.

In one or more embodiment(s), the input data of the reference spectra for machine learning may be in the form of a set of wavenumbers respectively associated with an intensity (or amplitude) value. For example, the set of wavenumbers may be comprised between 0 and 2,000 cm⁻¹ and according to a wavenumber step 0.1 cm⁻¹.

There are many machine learning techniques. All machine learning methods can be used to learn the rules relating the characteristics of the molasses and the performance of the yeasts. In one or more embodiment(s), the machine learning may in particular be based on the use of random forests (“random forest”), on the use of one or more linear regression algorithm(s), on the use of the K closest neighbours, on the use of deep learning (Deep learning), or the use of neural networks.

Furthermore, the machine learning may comprise, as input data, one or more hyperparameter(s). A hyperparameter is a parameter used to parameterise the algorithm (for example random forest, deep learning, etc.) before any learning. Hyperparameters may be specific to a type of machine learning and may therefore vary depending on the used type of machine learning.

For example, a hyperparameter may be the number of decision trees composing a random forest, the number of nodes in a tree, the size of the final nodes, or else the number of observations selected by “bootstrap” to build each tree.

After determining the model, the latter can be used afterwards to identify 205, for a current spectrum of a current molasses sample as input data 207, at least one spectral characteristic of the current spectrum, and determine a quality 209 of the current molasses.

Based on the determined quality of the current molasses, one or more performance(s) of a yeast produced using the current molasses as a nutrient source may be determined. The quality of the current molasses may be represented according to a class (for example poor, medium, good, very good, green light when it can be used or red light when it cannot be used or that it requires to be modified, etc.) or/and according to one or more score(s) relating to performances of the yeast, or/and a correlation between its spectral profile and an industrial performance statistical group.

Of course, the yeast performance obtained when the yeast is fed with the current molasses depends on several contributions such as the yeast strain used to produce the yeast, production conditions (for example pressure, temperature in the fermenter, configuration of the fermenter, etc.), and the inputs including the molasses used to feed the yeast production. In the context of the method of the present disclosure, it should be understood that the yeast performance(s) determined according to molasses qualities are determined for an identical yeast strain, identical production conditions, and identical inputs (except for the molasses).

In one or more embodiment(s), each reference spectrum of a respective molasses supplied for machine learning may have been obtained by characterising this respective molasses according to one or more optical measurement technique(s). For example, each reference spectrum may be representative of a combination of optical spectra of the same molasses and obtained by a respective optical measurement technique.

In one or more embodiment(s), the optical measurement technique on the molasses allowing obtaining the reference spectra is the same as that used on the current molasses to obtain the current spectrum. Furthermore, in one or more embodiment(s), the optical measurement technique used to obtain the spectra may consist of RAMAN spectroscopy.

In one or more embodiment(s), the data obtained after determining a molasses quality relating to a yeast performance according to the input spectrum 207 may be used to feed 210 the machine learning. Thus, advantageously, the statistical model can be improved continuously.

Of course, it is possible that the statistical model is not built every time it is requested to qualify an unknown molasses. The statistical model may be determined only once before being used with unknown spectra of molasses.

The random forest (“random forest”) methodology is described in more detail in the following publication:
Leo Breiman, Random Forests, Machine Learning, vol. 45, no 1, 2001, p. 5-32

As described before, the present disclosure is not limited to the use of random forest. Other types of machine learning may also be suitable, like, for example, the machine learning methods described in the following publications:
The Elements of Statistical Learning, 2nd edition, Trevor Hastie, Robert Tibshirani, Jerome Friedman, ISBN-13:978-0387848570
Deep Learning, Ian Goodfellow, Yoshua Bengio et Aaron Courville, ISBN-13:978-0262035613.

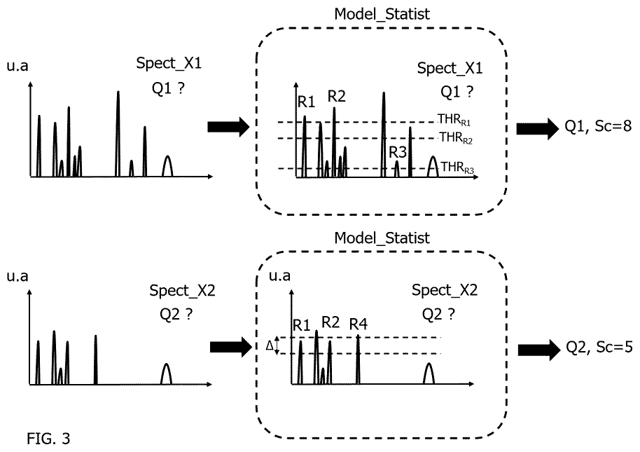

FIG. 3 illustrates an example of application of the statistical model obtained according to the method of the present disclosure on spectra of unknown molasses.

The respective quality (Q1; Q2) of two molasses M1 and M2 which could potentially be used as a source of nutrients in yeast production (for example yeast production by fermentation) may be desired.

This quality may be desired in order to know whether the molasses M1 and M2 could be used to feed a yeast production while allowing achieving one or more target performance(s) of the produced yeast, and/or in order to determine the most suitable molasses to produce a yeast having one or more target performance(s), or/and according to a trade-off of one or more target performance(s).

Referring to FIG. 3, for example, a discretisation between the two molasses M1 and M2 may be desired in order to determine the molasses that could lead to obtaining a yeast production yield higher than a value generally observed with a reference yeast performed in a reference method and/or obtaining a yeast (produced) that allow reaching specific volume values higher than a value generally observed with one reference yeast made in a reference method. According to another example, the discretisation between the two molasses M1 and M2 may be desired in order to determine the molasses that has the desired performance ratio, for example a production yield/a bread leavening capacity.

Each molasses M1; M2 may be associated respectively with an optical spectrum (Spect_X1 and Spect_X2), obtained for example by RAMAN spectroscopy.

Thus, the application of the statistical model (Model_statist) of molasses qualities on the molasses optical spectra (Spect_X1 and Spect_X2), different from the reference spectra having been used for machine learning, could allow identifying particular spectral characteristics. For example, the statistical model can determine a quality Q1 (and/or a score and/or a class) of the spectrum Spect_X1 based on the identification of the spectral characteristics corresponding to the presence of the wavenumbers R1, R2 and R3 coupled to a respective amplitude threshold TH_{R1}, TH_{R2}, TH_{R3} for each of the wavenumbers. Similarly, the prediction model can determine a quality Q2 of the spectrum Spect_X2 based on the identification of the spectral characteristics corresponding to the presence of the wavenumbers R1, R2 and R4 coupled to an amplitude range A to be complied with for the wavenumbers R1 and R2.

Based on the determined qualities (for example according to a score), the molasses M1 may seem to be a better candidate for feeding a yeast production than the molasses M2. Indeed, the molasses M1 has a score of 8, representative of a very good molasses, i.e. allowing, for example, obtaining a yeast production yield higher than a value generally observed with a reference yeast performed in a reference method, and/or obtaining a good bread leavening capacity defined by specific volume values that could be achieved higher than a value generally observed with a reference yeast performed in a reference method.

In one or more embodiment(s), for a plurality of molasses that could potentially be used to feed a yeast production (by fermentation), the molasses having a molasses quality higher than a predetermined quality (for example one or more score(s)) may be used to feed a yeast production. In one or more embodiment(s), among the molasses having a quality higher than a predetermined quality, the priority for feeding the yeast production may be given to the molasses having the best quality. For example, the best quality may be determined according to an ascending or descending order of the quality scores of the molasses or according to an ascending or descending order of the averages of the respective quality scores of each molasses.

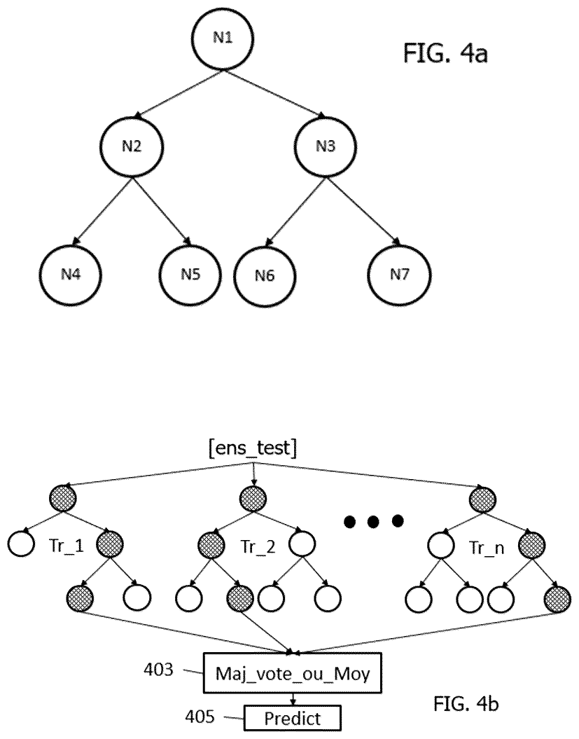

FIGS. 4a and 4b illustrate the principle of random forest for a statistical model in one or more embodiment(s).

The principle of the random forest algorithm consists in determining, according to a majority vote for categorical data or according to an average of individual results for quantitative data, the most probable prediction completed by a multitude of decision trees forming the random forest.

All of the decision trees of a random forest may be independent of one another, and the prediction determined by each decision tree before the majority vote or the establishment of the average could be decorrelated from the predictions determined by the other decision trees of the random forest.

A first step of this algorithm may consist in building decision trees (or decisional trees). FIG. 4a shows such a decision tree that could, for example, be divided into three portions:
a root node N1 corresponding to the first node of the tree (i.e. input node),
inner nodes N2; N3 which could have descendances (i.e. other nodes),
terminal nodes N4; N5; N6; N7 (also called sheets).

Each intermediate “node”, i.e. the nodes N2 and N3, may carry out a test relating to a variable whose result indicates the branch to be followed in the tree (i.e. N4, N5, N6 or N7). Thus, the test(s) performed at a node may define a partition rule for example.

The nodes N1 to N3 may be defined by explanatory variables (for example of the quantitative or qualitative type).

In order to build the different decision trees such as that shown in FIG. 4a, a selection of subsets of individuals from the set of training data with replacements may be performed thanks to a so-called “bootstrap” sampling, and which consists in randomly performing draws (with replacement) from individuals in the set of training data.

By replacement, it should be understood that the candidates selected in a subset remain in the training set, i.e. they can be selected for another subset. In general, the individuals used to build a decision tree are so-called “in bag” and the individuals used to assess the predictivity of the tree are generally so-called “out of bag” (referred to as “OOB”).

In turn, this set of training data may be obtained according to the set-up of a cross-validation strategy consisting in separating the initial set of data (for example 70/30) into two sub-sets of data, one for the training (for example 70%) and the other for testing (for example 30%) the prediction model.

Afterwards, each decision tree may be built (or trained) on a respective subset of individuals. This means that there may be as many subsets of individuals as decision trees, and the construction of the tree is performed only on one single subset of individuals.

In the case of the present disclosure, the initial set of data may correspond to a set of reference optical spectra (for example in the form of sets of wavenumbers) associated with a respective molasses quality (or/and one or more respective molasses score(s)) which could relate to at least one yeast performance (for example reference yeast) obtained when the yeast is fed with this molasses, and the respective variable used in the test of each node N1 to N3 may, for example, consist of an intensity (or amplitude) of a specific wavenumber.

During the training (or construction of the tree), at each construction of a new node, the variable enabling the partition at the node (in two descendant nodes) may be determined by the machine learning algorithm based on the random subset derived from the set of training data. For example, the machine learning may review (for example according to the discretisation step of the optical spectra) the set (or a subset) of the wavenumbers of the reference optical spectra of the random subset in order to determine a wavenumber and/or an intensity to be used as a variable. More specifically, the algorithm may seek to determine whether there is, for example, an intensity value allowing separating the spectra in the purest manner between the spectra corresponding to good-quality molasses or poor-quality molasses. For example, the test associated with the node N2 may consist in determining whether the amplitude (or intensity) associated with a particular wavenumber is higher than an intensity threshold or comprised within an intensity range.

The choice of one variable rather than another may be based on the measurement of the purity of the node using, for example, the Gini index or the Shannon entropy.

Thus, none of the input data supplied for machine learning influences it in its choice of the variables for partitioning at each node. Machine learning by random forest can herein determine its own variables (for example the wavenumbers, the amplitudes, etc.) in order to build a statistical model, with no a priori on the presence of one or more wavenumber(s) that should be detected in the reference spectra. For example, the node N3 may comprise a test based on an intensity value of a wavenumber that does not characterise any specific line or the presence of elements in particular (for example vitamin, nutrients, etc.) but having been determined simply as the wavenumber with its intensity value enabling the purest partition.

In turn, the terminal nodes N4 to N7 correspond to respective molasses qualities (or/and scores) and which may relate to the possible performances of a yeast.

For each decision tree, a prediction error (OOB error) may be calculated in order to determine the percentage of individuals poorly classified by the decision tree. The lower this rate and the more the decision tree could be considered as of good quality in its prediction.

As described before, most machine learning methods allow identifying the important factors that influence the predictions or the classifications the most. In the context of random forests, the algorithm may allow determining which specific intensities or wavenumber are important to discriminate the molasses. Several assessment methods have been described in the literature. For example, this importance may be determined by the number of times an intensity value associated with a wavenumber is used to partition the data at a node in the different decision trees of the random forest.

For example, the determination by the statistical model of some recurrences of intensity values associated with a given wavenumber may possibly inform on the specific presence of molecule or group of molecules like vitamins, nutrients or sugars according to a certain quantity (for example reflected by the amplitude/intensity associated with this wavenumber in RAMAN spectroscopy).

For example, a test based on an intensity value of a wavenumber that could characterise a line representative of a specific sugar may be implemented by a node of one or more decision tree(s) (of the random forest), another test based on an intensity value of a wavenumber that could characterise a line representative of a specific sugar may be implemented by a node of one or more decision tree(s), etc.

Advantageously, the determination of some recurrences could allow revealing a trend on the particular important elements allowing discretising a good molasses quality from a poor molasses quality.

FIG. 4 b illustrates an example of determination of a molasses quality by the statistical model for a reference optical spectrum as input data.

One or more reference optical spectrum/spectra derived from the set of test data [ens_test] previously presented may be provided as input of the statistical model based on a random forest trained as described before in order to test the relevance of its predictions at the model output.

For each reference optical spectrum supplied at the input of the model, the trees Tr_1; Tr_2; Tr_n can then respectively determine (grey nodes) a class, i.e. a score and/or a quality of the molasses of the input reference spectrum. Afterwards, the determination 405 of the molasses quality may be performed based on a majority vote 403 or according to the average in order to determine the most probable molasses quality and/or score. The molasses quality of the reference spectrum (for example RAMAN, or IR or THz spectrum) supplied as input being known, since it is derived from the set of test data, the accuracy of the statistical model based on the trained random forest may be determined by comparing the prediction of a quality obtained based on the model trained with the actual quality of the molasses.

Afterwards, the statistical model (Model_predict) having an acceptable prediction accuracy may be used to determine a quality and/or a score of an unknown molasses (for example Spect_X1 and spect_X2 of FIG. 3), the optical spectrum of which is supplied at the input of the model, and to deduce therefrom the performance(s) of a yeast produced by using the unknown molasses as a nutrient source.

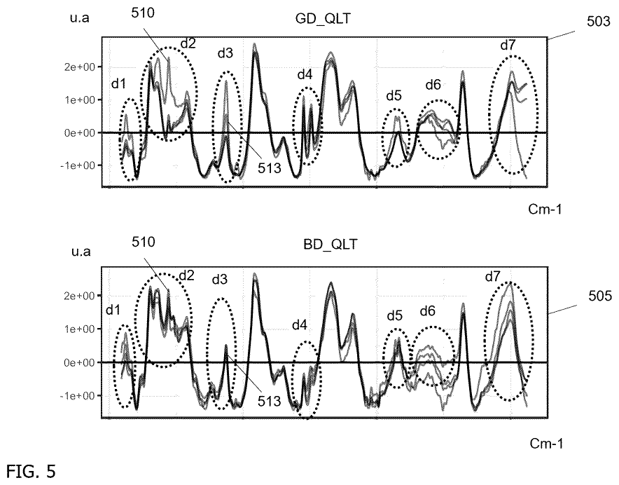

FIG. 5 shows, in one or more embodiment(s), different spectra of cane molasses of known quality obtained by RAMAN spectroscopy.

A Raman spectrum may be plotted as a function of the Raman shift in wavenumber and of its intensity in an arbitrary unit. The Raman shift corresponds to the difference between the light of the used monochromatic laser and the scattered light. The Raman intensity is the number of detected photons resulting from a Stokes Raman scattering. Indeed, in Raman spectroscopy, there are three types of scattering, Rayleigh scattering (elastic), Stokes Raman scattering (inelastic), and anti-Stoke Raman scattering (inelastic). In inelastic scattering, the photon having been emitted by a laser and having interacted with material is not re-emitted at the same wavelength, but with a Stokes or anti-Stokes shift. The Stokes Raman scattering is the most commonly used to obtain the optical spectrum of a sample.

Thus, four optical spectra (Stokes) 503 of cane molasses characterising good molasses quality (GD_QLT) and four optical spectra (Stokes) 505 of cane molasses characterising a poor molasses quality (BD_QLT) are depicted. Each spectrum may be representative of a molasses quality which allows obtaining a yeast with at least one respective performance (for example a given bread leavening capacity and/or a given yeast multiplication capacity). For example, these optical spectra may consist of reference spectra used to feed the machine learning by random forest described before.

Referring to FIG. 5, several areas of differences between a good and a poor molasses quality (d1 to d7) could be noticed. Nevertheless, except for the area d4 which has a marked difference between two respective molasses qualities, most of the other identified areas do not allow clearly determining the important criteria that could discretise a good molasses quality from a poor molasses quality, i.e. a molasses quality allowing obtaining a yeast having one or more acceptable performance(s). Indeed, based on the areas of differences d2 and/or d3 (or else d6), some molasses that have yet different quality may, however, have identical or very close spectra in some areas 510; 513.

In addition, while the area d4 seems to have marked differences when the molasses are of good or poor quality, the differences at this area could ultimately be barely or not relevant to be representative of a certain molasses quality (good or bad), and therefore representative of the performances of a yeast fed in production with these molasses of certain quality.

Thus, while a visual analysis of the optical spectra makes it very complicated to search for a correlation between a configuration of one or more spectral characteristic(s) and a molasses quality allowing obtaining a yeast having one or more acceptable performance(s), the advantageous use of the method as described in the present disclosure, i.e. a machine learning coupled with adequately selected input data (for example reference spectra, hyperparameters, etc.), allows obtaining a statistical model (for example based on a trained random forest) of effective molasses qualities in its prediction of the quality of molasses relating to yeast performances.

As mentioned before, obtaining a trained statistical model could then allow, before any yeast production, determining whether this molasses should be discarded or not because of poor quality (for example a score lower than 3 and/or poor class and/or red light), i.e. not allowing obtaining a yeast having one or more acceptable performance(s) (for example according to a specification set). Furthermore, based on the prediction of the quality or the quality score of the molasses, it may be possible to advantageously determine the nutrients and/or the proportions of nutrients to be used so as to compensate for the poor quality of a molasses.

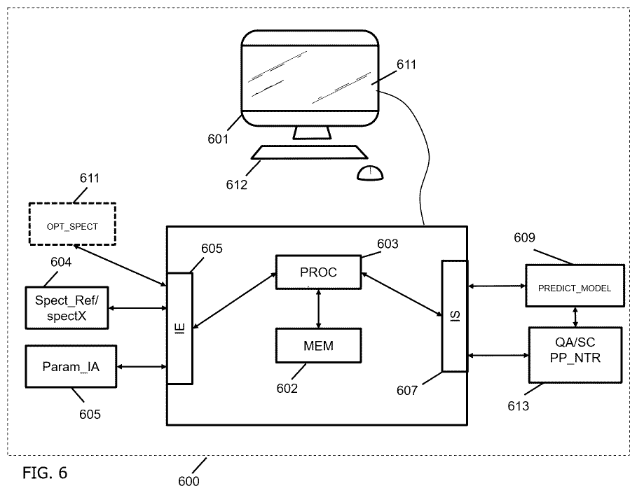

FIG. 6 shows, in one or more embodiment(s), a device for implementing the method of the present disclosure according to one or more embodiment(s).

In this embodiment, the device 600 may comprise a computer 601, this computer comprising a memory 602 for storing program instructions that could be loaded into a circuit, and able to cause the circuit 603 to execute the method of the present disclosure when the program instructions are managed by the circuit 603.

The memory 602 may also store data and information useful for carrying out the method of the present disclosure as described hereinabove.

For example, the circuit 603 may be:
a processor or a processing unit able to interpret instructions in a computer language, the processor or the processing unit may comprise, be associated with or be related to a memory comprising the instructions, or
associating a processor/processing unit and a memory, the processor or the processing unit adapted to interpret instructions in a computer language, the memory comprising said instructions, or
an electronic map in which the sequence of the method is described in silicon, or
a programmable electronic chip such as an FPGA (standing for “Field-Programmable Gate Array”) chip.

This computer may comprise an input interface 605 for receiving input data used for machine learning and an output interface for supplying a set of useful data. For example, the input interface may receive input data 604 such as reference optical spectra associated with a respective molasses quality for machine learning or spectra whose quality relating to at least one performance of a yeast should be determined. The input interface may also receive a set of configuration parameters 605 for configuring the machine learning, for example hyperparameters. Furthermore, optionally, the input interface may be connected 611 to a device enabling the optical measurement (for example RAMAN spectroscopy), or the device 600 may be directly integrated into an optical measurement device.

For example, the set of useful data supplied by the output interface 607 may be a trained prediction model 609, for example a statistical model of molasses quality relating to at least one yeast performance obtained from this molasses. Furthermore, the output interface may supply a prediction (or determination) 613 of a quality of a molasses (for example class and/or score) supplied 604 at the input interface 605, the molasses quality relating to at least one yeast performance obtained from this molasses. Finally, based on the trained prediction model, the output interface may also supply the nutrient(s) to be used and according to which proportions to correct a molasses quality determined by the trained prediction model.

To facilitate interaction with the computer 601, a screen 611 and a keyboard 612 may be provided for and connected to the computer circuit 603.

The expressions such as “comprise”, “include”, “incorporate”, “contain”, “be” and “have” should be interpreted in a non-exclusive manner when interpreting the description and its associated claims.

The method is not limited to the embodiments described hereinabove, only as example, but it encompasses all of the variants that a person skilled in the art could consider within the scope of the claims hereinafter.

Although described through a certain number of detailed embodiments, the proposed method and the device for implementing an embodiment of the method comprise different variants, modifications and improvements that will become apparent to a person skilled in the art, it being understood that these different variants, modifications and improvements are part of the scope of the present disclosure, as defined by the following claims. In addition, different aspects and features described hereinabove may be implemented together, or separately, or substituted with one another, and all of the different combinations and sub-combinations of the aspects and features are within the scope of the present disclosure. Furthermore, it is possible that some systems and equipment described hereinabove do not incorporate all of the modules and functions described for the preferred embodiments.

1-16. (canceled)

17. A method for qualifying molasses based on an optical measurement, the method comprising:
based on reference optical spectra of samples of distinct molasses, associated with respective known molasses qualities, building by machine learning a statistical model of molasses qualities as a function of at least one spectral characteristic of the reference spectra, and
for a current optical spectrum of a sample of a current molasses, based on the statistical model, identifying said at least one spectral characteristic of the current optical spectrum and determining a quality of the current molasses, said current molasses quality relating to at least one yeast performance obtained when said yeast is fed with said current molasses.

18. The method according to claim 17, wherein each molasses quality associated respectively with a distinct molasses sample relates to at least one yeast performance obtained when said yeast is fed with said distinct molasses sample.

19. The method according to claim 17, wherein said at least one performance is based on at least one criterion selected from among:
a multiplication capacity of the yeast fed with said current molasses,
a bread leavening capacity,
a capacity to be preserved,
a capacity to be dried,
a capacity to fix structural elements,
a capacity to assimilate nutrients,
a capacity to adapt to stresses.

20. The method according to claim 17, wherein the quality of the current molasses determined by said statistical model is defined by at least one molasses quality score, said at least one molasses quality score relating to said at least one performance.

21. The method according to claim 17, comprising:
illuminating a molasses sample with a first emitted light signal interacting with the sample, and collecting a second light signal, resulting from the interaction between the first light signal and the current sample; and
measuring the second light signal, in selected frequency bands, to establish an optical spectrum representative of a chemical signature of the current molasses sample.

22. The method according to claim 17, wherein the optical spectrum comprises a set of wavenumbers, one or more wavenumber(s) of the set of wavenumbers being representative of a chemical compound of the molasses.

23. The method according to claim 22, wherein the molasses includes compounds at least one from among a sugar, a mineral, a vitamin, and the optical spectrum includes a plurality of wavenumbers, and wherein at least one wavenumber characterises the presence of one of said compounds.

24. The method according to claim 17, wherein said at least one spectral characteristic is determined according to at least one criterion selected from among a presence or an absence of a predefined wavenumber, a position of a predefined number, an intensity value of a predefined wavenumber, overpassing of a threshold of an amplitude of a predefined wavenumber, a maximum or minimum intensity value of a predefined wavenumber, an intensity deviation between two predefined wavenumbers, an intensity of a predefined wavenumber comprised between two predetermined intensity values, or simple or complex combinations of the aforementioned spectral characteristics.

25. The method according to claim 17, wherein the optical measurement is carried out by at least one technique from among a RAMAN spectroscopy, an IR spectroscopy, or a THZ spectroscopy.

26. The method according to claim 17, wherein the range of values of the reference optical spectra is comprised between 1 cm⁻¹ and 2,500 cm⁻¹.

27. The method according to claim 25, wherein the range of values of the reference optical spectra is comprised between 436 cm⁻¹ and 1,700 cm⁻¹.

28. The method according to claim 17, wherein the molasses is a cane molasses or a beet molasses or a mixture of both, or a mixture of sugar products such as a molasses and a sugar syrup, or a mixture of products one of the components of which is the molasses.

29. A computer program comprising program code instructions for executing the method according to claim 17 when said program is executed on a computer.

30. A non-transitory computer-readable recording medium on which a program is recorded for implementing the method according to claim 17 when this program is executed by a processor.

31. A method for producing yeasts by fermentation comprising:
determining a respective molasses quality for each molasses of a plurality of molasses supplied at the input of the fermentation reaction according to the qualification method of claim 17, and
feeding the fermentation reaction with each molasse of the plurality of molasses whose respective molasses quality is higher than a predetermined quality, and for all or part of the molasses having a molasses quality lower than the predetermined quality, feeding the fermentation reaction after modification of the molasses, or/and discarding the molasses.

32. The method according to claim 31, wherein said modification of the molasses comprises at least one modification selected from among:
mixing the molasses having a molasses quality lower than the predetermined quality, with another molasses having a distinct molasses quality, and so that the molasses mixture has a molasses quality higher than the predetermined quality, and
adding one or more additive(s) so that the molasses improved by the additives has a molasses quality higher than the predetermined quality.
